(19) United States
(12) Patent Application Publication
(10) Pub. No.: US 2025/0057430 A1
(43) Pub. Date: Feb. 20, 2025

(54) PROBE FOR DETECTING AN OPENING SIZE OF AN OPENING IN A BLOOD VESSEL

(71) Applicant: Abiomed Europe GmbH, Aachen (DE)

(72) Inventors: Claudia MOURRAN, Aachen (DE); Thorsten SIESS, Aachen (DE)

(73) Assignee: Abiomed Europe GmbH, Aachen (DE)

(21) Appl. No.: 18/719,058

(22) PCT Filed: Dec. 13, 2022
(86) PCT No.: PCT/EP2022/085495

§ 371 Date: Jun. 12, 2024

(30) Foreign Application Priority Data
Dec. 15, 2021 (EP) 21214786.2

(51) Int. Cl. A61B 5/02; A61B 5/00; A61B 5/107

(52) U.S. Cl. CPC A61B 5/02007; A61B 5/1076; A61B 5/6847

(57) ABSTRACT

The present invention relates to a probe 10 for detecting an opening size of an opening in a blood vessel. The probe 10 comprises a first portion 12 having a first outer diameter D1, and a stepped portion 14 adjacent to the first portion 12. The stepped portion 14 comprises at least a first detection portion 16 and a second detection portion 18, the first detection portion 16 having a second outer diameter D2 and the second detection portion 18 having a third outer diameter D3. The second outer diameter D2 is larger than the first outer diameter D1 and smaller than the third outer diameter D3.

### BACKGROUND

The present invention relates to a probe for detecting a size or a diameter of an opening in a blood vessel.

Placement of catheters, all kind of heart pumps or other medical devices usually requires a percutaneous vascular access, for instance a vascular access to the femoral artery or a vascular access to the axillary artery. Generally, a minimally invasive approach is chosen to place said medical devices within the vascular system of the patient. Thus, the blood vessels to be accessed are not visible to the physician, but are covered by e.g. subcutaneous tissue, fascia and the skin. When removing the medical device again, the dilated and opened vessel is to be closed by a suitable closure device. The opening in the blood vessel, in particular in a vessel wall of the blood vessel, to be closed is sometimes also referred to as bore or puncture.

However, the size or diameter of the opening in the vessel is unknown to the physician. Although closure devices are available in different sizes, the physician has to choose an appropriate closure device on a “best guess” approach. In general, the physician will include the diameter of the vascular access and the diameter of the medical device to be placed or removed into his considerations for choosing an appropriate closure device.

Although the available information is helpful for the physician to choose the appropriate closure device, a further phenomenon can lead to distinct differences between the “best guess” approach of the physician and the actual size of the opening in the blood vessel, namely a so-called “recoil” of the blood vessel after dilation, sometimes also called elastic recoil capacity. Amongst others, this recoil of the dilated blood vessel depends on the presence of elastin in the wall of the blood vessel. The amount of elastin further varies by blood vessel and proximity to the aorta. Calcification of the blood vessel or other pathologic parameters will add another level of complexity and uncertainty to the size of the opening to be closed.

Accordingly, the unknown and variable recoil capacity of the blood vessel adds a distinct uncertainty to the approach of “best guess” based solely on the information on the devices used available to the physician. Further, the recoil capacity of a blood vessel is not yet subject of research and the studies at hand do show difference in the amount and expression of the recoil capacity of the blood vessels depending on a variety of internal and external factors. In particular, nearly all studies focus on elasticity measurements, e.g. Boutouyrie et al. (2009), Artery Research, 3, 3-8. To measure the elasticity of blood vessels a pulse wave analysis can be conducted. This measurement is helping to identify the general “age” of the blood vessel, however, it is not possible to derive any concrete numbers about the size of the opening in the blood vessel therefrom.

Therefore, there is the need to provide a tool to the physician which allows for an ameliorated educated guess of the actual size or diameter of the blood vessel or an opening in the blood vessel. The blood vessel may in particular be a artery. In addition, there is also the need to provide a tool for studying the recoil capacity of the openings in blood vessels during scientific research to increase the knowledge thereabout.

The present invention aims to solve this problem with a probe for detecting a size or a diameter of an opening in a blood vessel according to claim 1. Further embodiments are described in the dependent claims.

### SUMMARY OF THE INVENTION

The probe for detecting an opening size of an opening in a blood vessel comprises a first portion having a first outer diameter, and a stepped portion adjacent to the first portion. The stepped portion comprises at least a first detection portion and a second detection portion. The first detection portion has a second outer diameter, and the second detection portion has a third outer diameter. The second outer diameter is larger than the first outer diameter and smaller than the third outer diameter.

Accordingly, it is possible to reliably detect the opening size of an opening in the blood vessel. The opening size is the diameter or size of the opening in the blood vessel. Depending on which of the detection portions fully covering the size to be detected, the physician gets direct information on the approximate size to be detected.

Preferably, the stepped portion comprises n detection portions with n≥3, wherein each of the n detection portions have a (n+1)^{th} outer diameter and wherein the (n+1)^{th} outer diameter is larger than the n^{th} outer diameter. Thus, a plurality of detection portions is provided allowing for detection of a plurality of different sizes or diameters of an opening in a blood vessel.

Preferably, the probe has an elongated shape with a central axis. Preferably, a cross section of the probe at any position along the first portion and the stepped portion has a rounded shape and in particular a circular shape. Preferably, the probe has rotational symmetry, at least for the first portion and the stepped portion. Such a probe can easily be manufactured and can further be easily introduced into the blood vessel.

Preferably, the stepped portion further comprises a first transition portion between the first portion and the first detection portion, wherein the first transition portion extends non-perpendicular with respect to the central axis, wherein the first transition portion is preferably an oblique first transition portion slanted with respect to the first portion.

Preferably, the stepped portion further comprises a second transition portion between the first detection portion and the second detection portion, wherein the second transition portion extends non-vertical with respect to the central axis, wherein the second transition portion is preferably an oblique second transition portion slanted with respect to the first detection portion.

Preferably, the stepped portion further comprises a n^{th} transition portion between (n-1)^{th} detection portion and the n^{th} detection portion, wherein the n^{th} transition portion extends non-vertical with respect to the central axis, wherein the n^{th} transition portion is preferably an oblique n^{th} transition portion slanted with respect to the (n-1)^{th} detection portion.

Thus, the transition between the respective portions of the stepped portion is smoothened, so that the stepped portion can facilely be moved relative to the blood vessel and/or within the blood vessel.

Preferably, the probe is flexible, preferably elastically flexible. The probe preferably comprises at least one of PTFE, PTFEP, PVDF, FEP, PFA, ETFE, ECTFE, PPSU, PEEK, Forbon, Torlon, Vespel, Radel or UHMWPE on its outer surface and/or is at least partially composed of PTFE, PTFEP, PVDF, FEP, PFA, ETFE, ECTFE, PPSU, PEEK, Forbon, Torlon, Vespel, Radel or UHMWPE. Alternatively, a main body of the probe is made of at least one of PTFE, PTFEP, PVDF, FEP, PFA, ETFE, ECTFE, PPSU, PEEK, Forbon, Torlon, Vespel, Radel or UHMWPE on its outer surface and/or is at least partially composed of PTFE, PTFEP, PVDF, FEP, PFA, ETFE, ECTFE, PPSU, PEEK, Forbon, Torlon, Vespel, Radel or UHMWPE. This allows for a high flexibility and biocompatibility. Further, the materials allow for small coefficients of friction between the outer surface of the probe and the blood vessel.

Preferably, the probe further comprises a central opening, the central opening preferably extending through the entire probe. This allows to place the probe within a blood vessel by guiding the probe along a guidewire. Especially when placing heart pumps within a patient's heart, a guidewire is often introduced first into the blood vessel and the heart pump is guided along the guidewire for placement in the patient's heart. Accordingly, the probe can be guided along the identical guidewire to detect the diameter of the opening in the blood vessel and to support the physician in choosing a suitable closure device.

Preferably, the probe further comprises a sphere disposed on the first portion or adjacent to the first portion, the sphere having a sphere outer diameter larger than the first outer diameter, wherein the sphere is preferably a tapered sphere. The sphere has a defined sphere outer diameter which thus dilates the opening in the blood vessel by a predefined amount. As the first outer diameter of the first portion is smaller than the sphere outer diameter, the opening in the blood vessel may recoil again when the probe is pushed further forward into the blood vessel. When the stepped portion reaches the area initially dilated by the sphere, the diameter of the dilated area can be detected and hence, conclusions on the recoil capacity of the opening in the blood vessel can be drawn. Thus, the probe comprising a sphere is suitable for research regarding recoil capacity of blood vessels.

Preferably, the sphere outer diameter is identical to one of the second outer diameter, the third outer diameter or the (n+1)^{th} outer diameter of the respective detection portion. Hence, it can be evaluated if the opening in the blood vessel has recoiled when pushing the probe forward. For instance, in case the sphere outer diameter is identical to a fourth outer diameter of a third detection portion, it can be assumed that the third detection portion fully closes the blood vessel's opening in case no recoil occurred. However, in case recoil occurs, already the first detection portion or the second detection portion will fully close the opening in the blood vessel.

Preferably, the sphere is integrally formed with the first portion or configured to be detachably fixed to a free axial end of the first portion. Thus, a set of probes with different sphere outer diameters or different spheres having different sphere outer diameters may be provided to allow for a defined dilation of the blood vessel according to the specific needs for the particular blood vessel.

Preferably, the sphere comprises an introduction portion extending concentric with and away from the first portion, the introduction portion having an introduction portion outer diameter smaller than the sphere outer diameter, wherein the introduction portion outer diameter is preferably identical to the first outer diameter. The introduction portion facilitates the placement of the probe within the blood vessel.

Preferably, the probe comprises a sensing mechanism configured to detect liquid on an outer peripheral surface of the stepped portion. This allows to detect blood on the outer peripheral surface of the stepped portion and thus aids the physician in detecting the diameter of interest of the respective blood vessel.

Preferably, the sensing mechanism is configured to detect liquid on the outer peripheral surface of each of the detection portions independently. Based on the output of the sensing mechanism the physician recognizes which of the plurality of detection portions is currently fully closing the opening of interest of the blood vessel. For instance, in case the second detection portion fully closes the opening of interest of the blood vessel, liquid is detected only on the outer peripheral surface of the first detection portion and not on the outer peripheral surface of the second detection portion. Thus, the opening of interest of the blood vessel is fully closed by the second detection portion. The conclusion that the opening of interest of the blood vessel virtually corresponds to the third outer diameter may thus be drawn.

The sensing mechanism may comprise a first liquid sensor configured to detect liquid on the outer peripheral surface of the first detection portion and/or the sensing mechanism may comprise a second liquid sensor configured to detect liquid on the outer peripheral surface of the second detection portion and/or the sensing mechanism may comprise a n^{th} liquid sensor configured to detect liquid on the outer peripheral surface of the n^{th} detection portion.

Preferably, the number of liquid sensors equals the number of detection portions.

Alternatively, the sensing mechanism may comprise a first channel opening into the outer peripheral surface of the first detection portion, the first channel preferably extending through the probe in a direction away from the first portion. Liquid (i.e. blood) surrounding the outer peripheral surface of the first detection portion may enter the first channel and may cause a “flashback” of blood visualizing the physician that blood surrounds the first detection portion. Hence, the physician can draw the conclusion that the first detection portion does not fully close the opening of interest in the blood vessel.

The sensing mechanism may comprise a second channel opening into the outer peripheral surface of the second detection portion, the second channel extending through the probe in a direction away from the first portion. The sensing mechanism may comprise a n^{th} channel opening into the outer peripheral surface of the n^{th} detection portion, the n^{th} channel extending through the probe in a direction away from the first portion. Hence, in case for instance the third detection portion fully closes the opening of interest in the blood vessel, flashback occurs in the first channel and the second channel, but not in the third channel or any subsequent channel. Accordingly, the flashback of blood visualizes to the physician which detection portion fully closes the opening of interest in the blood vessel.

At least the stepped portion may be configured to be extendable, wherein the first detection portion may at least partially be disposed radially inwardly of the second detection portion, when the stepped portion is fully collapsed. The (n-1)^{th} detection portion may at least partially be disposed radially inwardly of the n^{th} detection portion. The stepped portion may thus be configured like a telescopic cylinder. To detect the recoil capacity of the blood vessel, the fully collapsed stepped portion is placed in the blood vessel or in the opening in the blood vessel respectively. Next, the outermost detection portion, i.e. the detection portion having the largest outer diameter, is drawn out of the blood vessel in that the stepped portion is partially expanded. In case liquid surrounds the then radially outermost detection portion no or nearly no recoil of the blood vessel or the opening in the blood vessel has occurred. In case no liquid surrounds the then outermost detection portion, recoil has occurred. Thus, the stepped portion is extended one step further in that the next detection portion is drawn out of the blood vessel and it is again monitored if liquid surrounds the then outermost detection portion or not.

Preferably, the third outer diameter is at least one French larger than the second outer diameter. Preferably, the (n+1)^{th} outer diameter is at least on French larger than the n^{th} outer diameter. The French scale is commonly used to measure the size of medical devices, such as catheters or closure devices. Using steps of one French is advantageous, as the so detected opening size of an opening in the blood vessel can directly be transferred for choosing the correct closure device. Of course, it is also possible to use a different scale or different steps of e.g. 0.5 French or 1.5 French.

### BRIEF DESCRIPTION OF THE DRAWINGS

The foregoing summary, as well as the following detailed description of exemplary embodiments, will be better understood when read in conjunction with the appended drawings. For the purpose of illustrating the present disclosure, reference is made to the drawings. The scope of the disclosure is not limited, however, to the specific embodiments disclosed in the drawings.

In the drawings:

FIG. 1 is a schematic view of blood vessels of a human body,

FIG. 2 is a schematic side view of a probe according to a first embodiment,

FIG. 3 is a schematic cross section of a probe having a sensing mechanism according to a first embodiment, and

FIG. 4 is a schematic cross section of a probe having a sensing mechanism according to a second embodiment.

FIG. 5 is a schematic side view of a probe according to a second embodiment,

FIG. 6a is a first schematic side view of a probe according to a third embodiment, and

FIG. 6b is a second schematic side view of the probe shown in FIG. 6a.

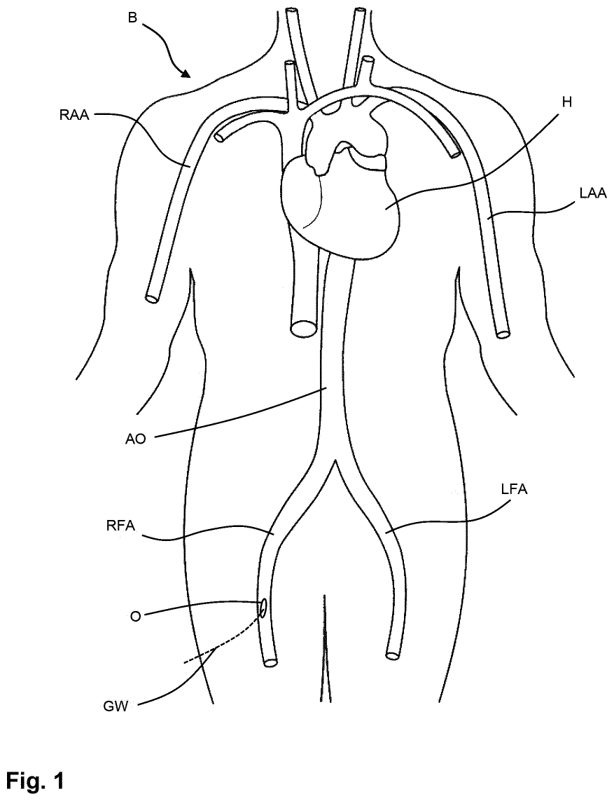

FIG. 1 depicts a schematic view of a human body B. Here, the right axillary artery RAA, the left axillary artery LAA, the right femoral artery RFA and the left femoral artery LFA are shown schematically. The arteries open into the aorta AO, which opens into the heart H. For placement of a medical device within the vascular system or the heart, a vascular access is needed. Therefore, the physician percutaneously introduces an introducer sheath through an opening O in a blood vessel into the vascular system. In the depicted example of FIG. 1, the opening O is provided in the right femoral artery RFA and the medical device to be placed may be a heart pump to assist or replace the natural cardiac pump function by a continuous pumping operation. Commonly, such heart pumps are placed using a minimally invasive approach.

For placement of a heart pump, such as a catheter heart pump or intravascular heart pump, the physician usually introduces a guide wire GW through the opening O in the right femoral artery RFA and pushes the same forward through the ascending aorta AO into the left ventricle of the heart H. The heart pump is then guided along the guide wire GW through the ascending aorta AO to the left ventricle of the heart H. Therefore, the heart pump has to pass the opening O in the right femoral artery RFA which has to have a diameter sufficiently large to allow for unobstructed introduction of the heart pump into the right femoral artery RFA

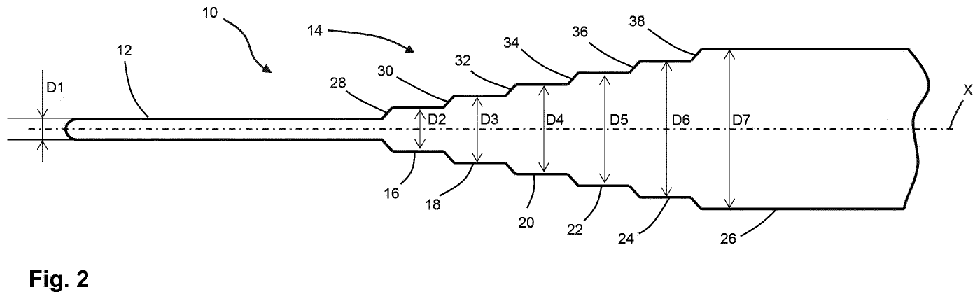

After the heart pump or the medical device has been removed from the patient's heart H, the opening O needs to be closed, for example by a suitable closing device. Due to the minimally invasive approach, the opening O is not visible as being covered by subcutaneous tissue, facias and the skin of the patient. For evaluation the size or diameter of the opening O, the physician may use a probe 10, 100, 110 as schematically shown in FIGS. 2 to 6b, as explained in more detail in the following.

FIG. 2 schematically shows a side view of a probe 10 according to a first embodiment. The probe 10 comprises a main body with a first portion 12 and a stepped portion 14. The probe 10 has an elongated shape and a central axis X. In the embodiment shown, the probe 10 has rotational symmetry and the main body of the probe is entirely composed of an elastically flexible material, such as PTFE. Of course, the main body of the probe 10 may also be composed of another material or may be composed of a base material coated by a second material.

The stepped portion 14 is directly adjacent to the first portion 12. The first portion 12 has a first outer diameter D1 of e.g. 8 French. In this embodiment, the stepped portion comprises six detection portions 16-26. The first detection portion 16 has a second outer diameter D2 of e.g. 9 French. The second detection portion 18 has a third outer diameter D3 of e.g. 10 French. The third detection portion 20 has a fourth outer diameter D4 of e.g. 11 French. The fourth detection portion 22 has a fifth outer diameter D5 of e.g. 12 French. The fifth detection portion 24 has a sixth outer diameter D6 of e.g. 13 French and the sixth detection portion 26 has a seventh outer diameter D7 of e.g. 14 French. Although six detection portions 18-26 are shown, the probe 10 may also have only two detection portions or more than six detection portions. Further, the diameters of the detection portions may also vary in steps of e.g. 0.5 French or 2 French.

The stepped portion 14 further comprises an oblique first transition portion 28 connecting the first portion 12 and the first detection portion 16. The first transition portion 28 is slanted with respect to the first portion 12 and thus extends non perpendicular with respect to the central axis X. Accordingly, the first transition portion 28 form a continuous portion smoothly connecting the first portion 12 and the first detections portion 16. Analogously, the stepped portion comprises an oblique second transition portion 30 smoothly connecting the first detection portion 16 and the second detection portion 18, an oblique third transition portion 32 smoothly connecting the second detection portion 18 and the third detection portion 20, an oblique fourth transition portion 34 smoothly connecting the third detection portion 20 and the fourth detection portion 22, an oblique fifth transition portion 36 smoothly connecting the fourth detection portion 22 and the fifth detection portion 24, and a sixth transition portion 38 smoothly connecting the fifth detection portion 24 and the sixth detection portion 26.

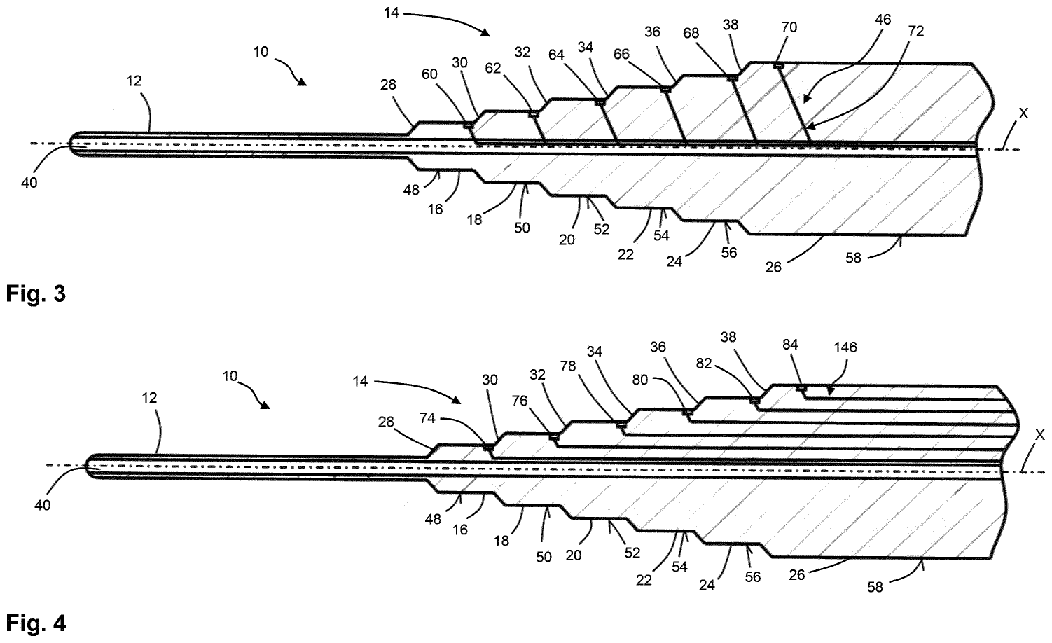

The probe 10 further comprises a central opening 40, as shown in FIGS. 3 and 4. The central opening 40 is configured to receive the guidewire GW so that the probe 10 can be placed inserted into the opening O in the blood vessel by guiding the probe 10 along the guidewire GW.

The axial extension of each of the first detection 16 portion to the fifth detection portion 24 may be equal and may e.g. be 10 French. The sixth detection portion 26 may extend axially along the remaining probe 10 up to the axial end opposite of the first portion 12. Of course, a further portion may be provided next to the sixth detection portion 26 having a diameter different from the seventh outer diameter D7 of the sixth detection portion 26. The axial extension of the first portion 12 may be e.g. 15 to 20 French. Further, the first portion 12 may comprise a spherical free axial end, which facilitates introduction of the probe 10 into the opening O in the blood vessel.

Further, the probe 10 may comprise a sensing mechanism 46, 146 configured to detect liquid on an outer peripheral surface of the stepped portion 14. In particular, the sensing mechanism 46, 146 is configured to detect liquid (i.e. blood) on the outer peripheral surface of each of the detection portions 16-26.

FIG. 3 depicts a first embodiment of the sensing mechanism 46. The sensing mechanism 46 comprises a first liquid sensor 60 configured to detect liquid (i.e. blood) surrounding the outer peripheral surface 48 of the first detection portion 16, a second liquid sensor 62 configured to detect liquid (i.e. blood) surrounding the outer peripheral surface 50 of the second detection portion 18, a third liquid sensor 64 configured to detect liquid (i.e. blood) surrounding the outer peripheral surface 52 of the third detection portion 20, a fourth liquid sensor 66 configured to detect liquid (i.e. blood) surrounding the outer peripheral surface 54 of the fourth detection portion 22, a fifth liquid sensor 68 configured to detect liquid (i.e. blood) surrounding the outer peripheral surface 56 of the fifth detection portion 24 and a sixth liquid sensor 70 configured to detect liquid (i.e. blood) surrounding the outer peripheral surface 58 of the sixth detection portion 26.

As shown in FIG. 3, each of the liquid sensors 60-70 is arranged to be flush with the outer peripheral surface 48-58 of the respective detection portions 16-26. The liquid sensors 60-70 are configured to detect the presence of liquid (i.e. blood) surrounding the respective outer peripheral surface 48-58 of the respective detection portion 16-26 independently from each other. In other words, when the probe 10 is placed within the blood vessel and if blood surrounds only the first detection portion 16 and the second detection portion 18, but none of the remaining detection portions 20-26, a respective signal will be emitted indicating to the physician that blood is only surrounding the outer peripheral surfaces 48, 50 of the first detection portion 16 and the second detection portion 18.

For transmitting the emitted signals, the liquid sensors 60-70 are connected to a cable loom 72 for transmitting the signals. The cable loom 72 may comprise a single central cable guided along the central opening 40. However, the cable loom 72 may have any suitable design.

When the probe 10 introduced into an opening O of a blood vessel, such as the right femoral artery RFA, blood surrounds the outer peripheral surfaces 48-58 of the detection portions 16-26. The presence of blood will be detected by the liquid sensors 60-70. In case one of the detection portions 16-26 fully closes the opening size of an opening of interest in the blood vessel, no signal will be emitted for the respective detection portion and all subsequent (i.e. bigger) detection portions. Thus, the physician then can directly draw conclusions on the opening size or diameter of interest of e.g. the opening O of the blood vessel, as the diameter D2-D7 of the respective detection portion 16-26 fully closing the size or diameter of interest is then known to the physician.

FIG. 4 depicts a second embodiment of the sensing mechanism 146. The sensing mechanism 146 according to the second embodiment comprises a plurality of channels 74-84. In particular, the sensing mechanism 146 comprises a first channel 74 opening into the outer peripheral surface 48 of the first detection portion 16. The first channel 74 extends through the probe 10 in a direction away from the first portion 12 in parallel to the central axis X. The sensing mechanism 146 comprises a second channel 76 opening into the outer peripheral surface 50 of the second detection portion 18. The second channel 76 extends through the probe 10 in a direction away from the first portion 12 in parallel to the central axis X. The sensing mechanism 146 comprises a third channel 78 opening into the outer peripheral surface 52 of the third detection portion 20. The third channel 78 extends through the probe 10 in a direction away from the first portion 12 in parallel to the central axis X. The sensing mechanism 146 comprises a fourth channel 80 opening into the outer peripheral surface 54 of the fourth detection portion 22. The fourth channel 80 extends through the probe 10 in a direction away from the first portion 12 in parallel to the central axis X. The sensing mechanism 146 comprises a fifth channel 82 opening into the outer peripheral surface 56 of the fifth detection portion 24. The fifth channel 82 extends through the probe 10 in a direction away from the first portion 12 in parallel to the central axis X. The sensing mechanism 146 comprises a sixth channel 84 opening into the outer peripheral surface 58 of the sixth detection portion 26. The sixth channel 84 extends through the probe 10 in a direction away from the first portion 12 in parallel to the central axis X.

All channels 74-84 are configured to allow for blood flow therethrough. Accordingly, when the probe 10 comprising the sensing mechanism 146 according to the second embodiment is introduced into the blood vessel, blood may flow through the channels 74-84 due to the prevailing blood pressure within the blood vessel. The respective “flashback” occurring at the end of the respective channels 74-84 indicates to the physician which detection portion 16-26 is surrounded by blood. In case no “flashback” of blood occurs for some or all of the channels 74-84, the physician can directly draw conclusions on the opening size or diameter of interest of e.g. the opening O of the blood vessel, as the diameter D2-D7 of the respective detection portion 16-26 fully closing the opening size or diameter of interest is then known to the physician.

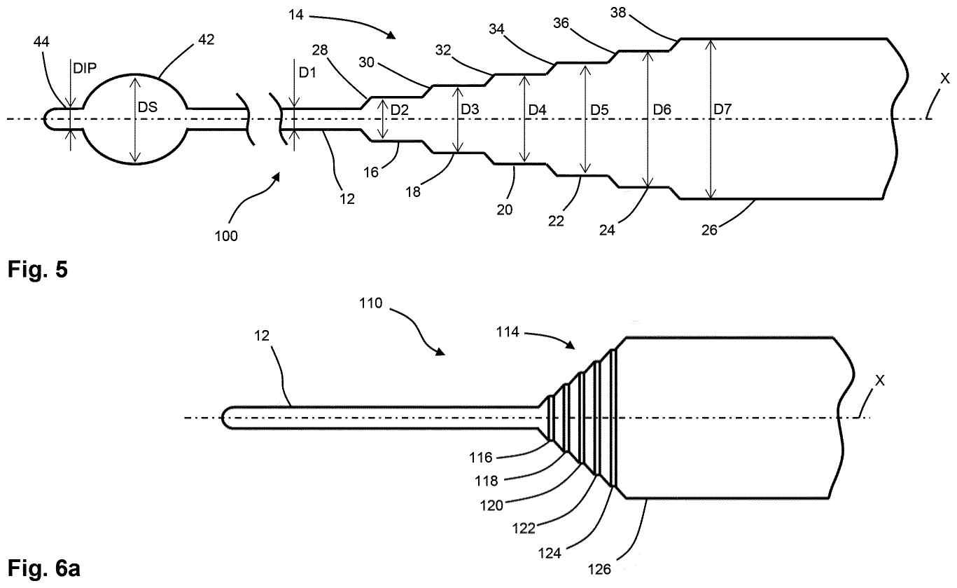

FIG. 5 depicts a schematic side view of a probe 100 according to a second embodiment. The probe 100 according to the second embodiment differs from the probe 10 according to the first embodiment in that a tapered sphere 42 is provided. In this embodiment, the tapered sphere 42 is disposed on the first portion 12 integrally formed with the first portion 12. Of course, the sphere 42 may also be configured to be detachable, e.g. via respective threading.

The sphere 42 is tapered so that it has an oval-like shape when viewed from the side. Further, the sphere 42 comprises an introduction portion 44 extending concentric with and away from the first portion 12. The introduction portion 44 has an introduction portion outer diameter DIP identical to the first outer diameter D1 of the first portion 12. Further, the introduction portion 44 has a spherical shape at its free axial end to facilitate introduction of the probe 100 into the blood vessel.

The sphere 42 has a sphere outer diameter DS larger than the first diameter D1. In this embodiment, the sphere 42 has a sphere outer diameter DS identical to the fourth outer diameter D4 of the third detection portion 20, e.g. 11 French.

Of course, the probe 100 according to the second embodiment may comprises a sensing mechanism 46, 146 as shown in FIGS. 3 and 4.

Although the probe 100 according to the second embodiment may be used analogously to the probe 10 according to the first embodiment, the main purpose of the probe 100 according to the second embodiment is to evaluate the recoil capacity of an opening O of a blood vessel. Therefore, the probe 100 is introduced into the blood vessel and the sphere 42 dilates the opening O of the blood vessel with a known diameter, i.e. the sphere outer diameter DS. Accordingly, the sphere outer diameter 42 needs to be sufficiently large to dilate the opening O of the blood vessel.

When the probe 100 is pushed further into the blood vessel, one of the detection portions 16-26 will fully close the opening O of the blood vessel. In case a detection portion 16-26 having the identical outer diameter D2-D7 as the sphere outer diameter DS fully closes the opening O of the blood vessel, likely no recoil has occurred. In case a detection portion 16-26 having a smaller outer diameter D2-D7 as the sphere outer diameter DS fully closes the blood vessel, a certain amount of recoil has occurred, namely the difference between the sphere outer diameter DS and of the outer diameter D2-D7 of the detection portion 16-26 fully closing the opening O of the blood vessel. Hence, the physician can then draw conclusions on the recoil capacity of the opening O of the blood vessel.

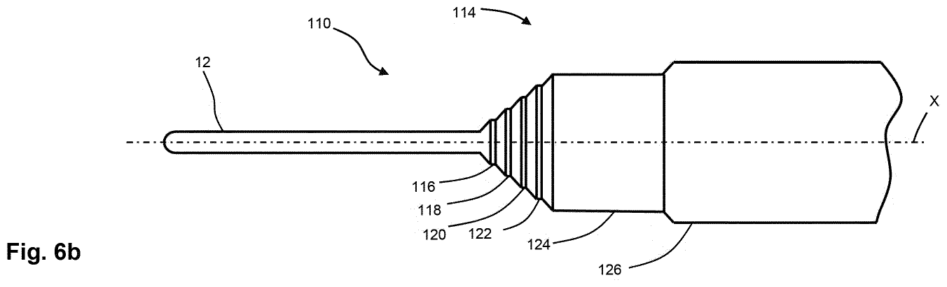

FIGS. 6a and 6b depict a probe 110 according to a third embodiment. In this embodiment, the stepped portion 114 differs from the probe 10 according to the first embodiment and from the probe 100 according to the second embodiment. The stepped portion 114 is configured to be extendable, in particular in a direction parallel to the central axis X. In particular, the detection portions 116-126 are collapsible relative to each other so that stepped portion 114 is configured like a telescopic cylinder, see FIG. 6a. In particular, in the fully collapsed state the first detection portion 116 is at least partially disposed radially inwardly of the other detection portions 118-126. Accordingly, the second detection portion 118 is at least partially disposed radially inwardly of the third detection portion 120 and the subsequent detection portions 122-126 in the fully collapsed state of the stepped portion 114. The third detection portion 120 is at least partially disposed radially inwardly of the fourth detection portion 122, the fifth detection portion 124 and the sixth detection portion 126. The fourth detection portion 122 is at least partially disposed radially inwardly of the fifth detection portion 124 and the sixth detection portion. The fifth detection portion 124 is at least partially disposed radially inwardly of the sixth detection portion 126. The sixth detection portion 126 is the radial outermost detection portion.

To evaluate the recoil capacity of an opening O of a blood vessel, the probe 110 is placed within the blood vessel in the fully collapsed state thereby dilating the opening O of the blood vessel, as the sixth detection portion 126 is disposed in the opening O of the blood vessel and has a larger outer diameter D7 than the opening O of the blood vessel. Next, the stepped portion 114 is extended detection portion by detection portion. First, the sixth detection portion 126 is moved relative to the remaining detection portions 116-124 and the first portion 12. A partially extended stepped portion 114 with the sixth detection portion 126 moved relative to the remaining detection portions is shown in FIG. 6b.

If recoil of the opening O of the blood vessel occurs, the fifth detection portion 124 then fully closes the opening O of the blood vessel. If so, the stepped portion 114 is further extended in moving the fifth detection portion 124 and the sixth detection portion 126 relative to the remaining detection portions and the first portion 12. If again recoil of the opening O of the blood vessel occurs, the fourth detection portion 122 fully closes the opening O of the blood vessel. The physician continues to extend the stepped portion 114 detection portion by detection portion until blood flow is detected, e.g. with a sensing mechanism 46, 146 as described above. This allows to directly draw conclusions on the recoil capacity of the opening O of the blood vessel.

It has to be noted that above-described embodiments of the probes 10, 100, 110 and sensing mechanisms 46, 146 can be combined. For instance, the probe 110 comprising an extendable stepped portion 114 may additionally comprise a sphere 42 and/or a sensing mechanism 46, 146.

### EXEMPLARY IMPLEMENTATIONS

As already described, the technology described herein may be implemented in various ways. In that regard, the foregoing disclosure is intended to include, but not be limited to, the systems, methods, and combinations and sub-combinations thereof that are set forth in the following exemplary implementations. Preferred embodiments are described in the following paragraphs:

A1 Probe for detecting an opening size of an opening in a blood vessel comprising:
a first portion having a first outer diameter,
and a stepped portion adjacent to the first portion, the stepped portion comprising at least a first detection portion and a second detection portion, the first detection portion having a second outer diameter and the second detection portion having a third outer diameter,
wherein the second outer diameter is larger than the first outer diameter and smaller than the third outer diameter.

A2 Probe according to paragraph A1, wherein the stepped portion comprises n detection portions with n≥3, wherein each of the n detection portions have a (n+1)^{th} outer diameter and wherein the (n+1)^{th} outer diameter is larger than the n^{th} outer diameter.

A3 Probe according to paragraph A1 or A2, wherein the probe has an elongated shape with a central axis.

A4 Probe according to paragraph A3, wherein the stepped portion further comprises a first transition portion between the first portion and the first detection portion, wherein the first transition portion extends non-perpendicular with respect to the central axis wherein the first transition portion is preferably an oblique first transition portion slanted with respect to the first portion.

A5 Probe according to paragraph A3 or A4, the stepped portion further comprises transition portions between each of the detection portions, wherein the transition portions extend non-perpendicular with respect to the central axis, wherein the transition portions are preferably oblique transition portions slanted with respect to the detection portions.

A6 Probe according to any one of the preceding paragraphs A1 to A5, wherein the probe is flexible, preferably elastically flexible.

A7 Probe according to any one of the preceding paragraphs A1 to A6, wherein the probe further comprises a central opening, the central opening preferably extending through the entire probe.

A8 Probe according to any one of the preceding paragraphs, wherein the probe further comprises a sphere disposed on the first portion or adjacent to the first portion, the sphere having a sphere outer diameter larger than the first outer diameter, wherein the sphere is preferably a tapered sphere.

A9 Probe according to paragraph A8, wherein the sphere is integrally formed with the first portion or configured to be detachably fixed to a free axial end of the first portion.

A10 Probe according to paragraph A8 or A9, wherein the sphere comprises an introduction portion extending concentric with and away from the first portion, the introduction portion having an introduction portion outer diameter smaller than the sphere outer diameter, wherein the introduction portion outer diameter is preferably identical to the first outer diameter.

A11 Probe according to any one of the preceding paragraphs, wherein the probe comprises a sensing mechanism configured to detect liquid on an outer peripheral surface of the stepped portion.

A12 Probe according to paragraph A11, wherein the sensing mechanism is configured to detect liquid on the outer peripheral surface of each of the detection portions independently.

A13 Probe according to paragraph A11 or A12, wherein the sensing mechanism comprises liquid sensors configured to detect liquid on the outer peripheral surface of each of the detection portions, wherein the number of liquid sensors preferably equals the number of detection portions.

A14 Probe according to any one of the paragraphs A11 to A13, wherein the sensing mechanism comprises a first channel opening into the outer peripheral surface of the first detection portion, the first channel preferably extending through the probe in a direction away from the first portion.

A15 Probe according to any one of the paragraphs A11 to A14, wherein the sensing mechanism comprises a second channel opening into the outer peripheral surface of the second detection portion, the second channel preferably extending through the probe in a direction away from the first portion.

A16 Probe according to any one of the paragraphs A11 to A15, wherein the sensing mechanism may comprise a n^{th} channel opening into the outer peripheral surface of the n^{th} detection portion, the n^{th} channel preferably extending through the probe in a direction away from the first portion.

A17 Probe according to any one of the preceding claims, wherein at least the stepped portion is configured to be extendable, wherein the first detection portion is preferably at least partially disposed radially inwardly of the second detection portion, when the stepped portion is fully collapsed.

A18 Probe according to any one of the preceding claims, wherein the third outer diameter is at least one French larger than the second outer diameter.

A19 Probe according to any one of the preceding claims, wherein the probe comprises at least one of PTFE, PTFEP, PVDF, FEP, PFA, ETFE, ECTFE, PPSU, PEEK, Forbon, Torlon, Vespel, Radel or UHMWPE on its outer surface and/or is at least partially composed of PTFE, PTFEP, PVDF, FEP, PFA, ETFE, ECTFE, PPSU, PEEK, Forbon, Torlon, Vespel, Radel or UHMWPE or wherein a main body of the probe is made of at least one of PTFE, PTFEP, PVDF, FEP, PFA, ETFE, ECTFE, PPSU, PEEK, Forbon, Torlon, Vespel, Radel or UHMWPE on its outer surface and/or is at least partially composed of PTFE, PTFEP, PVDF, FEP, PFA, ETFE, ECTFE, PPSU, PEEK, Forbon, Torlon, Vespel, Radel or UHMWPE.

A20 Probe according to any one of the preceding paragraphs A1 to A19, wherein a cross section of the probe at any position along the first portion and the stepped portion has a rounded shape and in particular a circular shape.

A21 Probe according to any of the preceding paragraphs A1 to A20, wherein at least the first portion and the stepped portion of the probe have rotational symmetry.

A22 Method for detecting an opening size of an opening in a blood vessel with a probe according to any one of paragraphs A1 to A22, wherein the method comprises the following steps:
placing the probe within the blood vessel;
moving the stepped portion relative to the blood vessel; and
detecting blood flow while moving the stepped portion.

### LIST OF REFERENCE SIGNS

10, 100, 110 probe
12 first portion
14, 114 stepped portion
16, 116 first detection portion
18, 118 second detection portion
20, 120 third detection portion
22, 122 fourth detection portion
24, 124 fifth detection portion
26, 126 sixth detection portion
30 first transition portion
32 second transition portion
34 third transition portion
36 fourth transition portion
38 fifth transition portion
40 central opening
42 sphere
44 introduction portion
46, 146 sensing mechanism
48 outer peripheral surface of first detection portion
50 outer peripheral surface of second detection portion
52 outer peripheral surface of third detection portion
54 outer peripheral surface of fourth detection portion
56 outer peripheral surface of fifth detection portion
58 outer peripheral surface of sixth detection portion
60 first liquid sensor
62 second liquid sensor
64 third liquid sensor
66 fourth liquid sensor
68 fifth liquid sensor
70 sixth liquid sensor
72 cable loom
74 first channel
76 second channel
78 third channel
80 fourth channel
82 fifth channel
84 sixth channel
AO aorta
B body
D1 first outer diameter
D2 second first outer diameter
D3 third first outer diameter
D4 fourth first outer diameter
D5 fifth first outer diameter
D6 sixth first outer diameter
D7 seventh first outer diameter
DIP introduction portion outer diameter
DS sphere outer diameter
GW guidewire
H heart
LAA left axillary artery
LFA left femoral artery
O opening
RAA right axillary artery
RFA right femoral artery
X central axis

1. Probe for detecting an opening size of an opening in a blood vessel comprising:
a first portion having a first outer diameter (D1),
and a stepped portion adjacent to the first portion, the stepped portion comprising at least a first detection portion and a second detection portion, the first detection portion having a second outer diameter (D2) and the second detection portion having a third outer diameter (D3),
wherein the second outer diameter (D2) is larger than the first outer diameter (D1) and smaller than the third outer diameter (D3).

2. Probe according to claim 1, wherein the stepped portion comprises n detection portions with n≥3, wherein each of the n detection portions have a (n+1)^{th} outer diameter (D4-D7) and wherein the (n+1)^{th} outer diameter (D4-D7) is larger than an n^{th} outer diameter (D3-D6).

3. Probe according to claim 1, wherein the probe has an elongated shape with a central axis (X).

4. Probe according to claim 3, wherein the stepped portion further comprises a first transition portion between the first portion and the first detection portion, wherein the first transition portion extends non-perpendicular with respect to the central axis (X), wherein the first transition portion is preferably an oblique first transition portion slanted with respect to the first portion.

5. Probe according to claim 3, wherein the stepped portion further comprises transition portions between each of the detection portions, wherein the transition portions extend non-perpendicular with respect to the central axis (X), wherein the transition portions are preferably oblique transition portions slanted with respect to the detection portions.

6. Probe according to claim 1, wherein the probe is flexible, preferably elastically flexible.

7. Probe according to claim 1, wherein the probe further comprises a central opening, the central opening preferably extending through the entire probe.

8. Probe according to claim 1, wherein the probe further comprises a sphere disposed on the first portion or adjacent to the first portion, the sphere having a sphere outer diameter (DS) larger than the first outer diameter (D1), wherein the sphere is preferably a tapered sphere.

9. Probe according to claim 8, wherein the sphere is integrally formed with the first portion or configured to be detachably fixed to a free axial end of the first portion.

10. Probe according to claim 8, wherein the sphere comprises an introduction portion extending concentric with and away from the first portion, the introduction portion having an introduction portion outer diameter (DIP) smaller than the sphere outer diameter (DS), wherein the introduction portion outer diameter (DIP) is preferably identical to the first outer diameter (D1).

11. Probe according to claim 1, wherein the probe comprises a sensing mechanism configured to detect liquid on an outer peripheral surface of the stepped portion.

12. Probe according to claim 11, wherein the sensing mechanism is configured to detect liquid on the outer peripheral surface each of the detection portions independently.

13. Probe according to claim 11, wherein the sensing mechanism comprises liquid sensors configured to detect liquid on the outer peripheral surface of each of the detection portions, wherein the probe wherein a number of liquid sensors preferably equals the number of detection portions.

14. Probe according to claim 1, wherein at least the stepped portion is configured to be extendable, wherein the first detection portion is preferably at least partially disposed radially inwardly of the second detection portion, when the stepped portion is fully collapsed.

15. Probe according to claim 1, wherein the third outer diameter (D3) is at least one French larger than the second outer diameter (D2).
